(19) United States
(12) Patent Application Publication
(10) Pub. No.: US 2002/0146409 A1
(43) Pub. Date: Oct. 10, 2002

(54) Methods for stabilizing lyophilized blood proteins

(76) Inventors: Steven W. Herring, San Dimas, CA (US); Daniel M. Chavez, Alta Loma, CA (US); John A. Morris, Glendale, CA (US)

Correspondence Address: CHRISTIE, PARKER & HALE, LLP, 350 WEST COLORADO BOULEVARD, SUITE 500, PASADENA, CA, 91105, US

(21) Appl. No.: 09/772,634

(22) Filed: Jan. 30, 2001

(51) Int. Cl. A61K 38/48; A61K 31/715

(52) U.S. Cl. 424/94.64; 514/21; 514/58

(57) ABSTRACT

Methods for stabilizing lyophilized blood proteins, preferably fibrinogen, comprise forming a stable complex between the blood protein and hydroxypropyl-α-cyclodextrin in an aqueous solution. Hydroxypropyl-α-cyclodextrin is added to the blood protein in an amount sufficient to form a stable complex with the protein. The solution is lyophilized to form a lyophilized protein/hydroxypropyl-α-cyclodextrin complex. The lyophilized protein/hydroxypropyl-α-cyclodextrin complex is then reconsitituted.

### FIELD OF THE INVENTION

The present invention relates to methods for stabilizing lyophilized blood proteins using hydroxypropyl-α-cyclodextrin.

### BACKGROUND OF THE INVENTION

Fibrinogen is an important blood protein. Fibrinogen-containing solutions can be infused intravenously as replacement therapy for afibrogenemic patients. They are also a component of fibrin glue (FG) preparations. FG contains two components, fibrinogen and thrombin, which, when mixed together, form a “glue” for wound closure or for producing hemostasis at an injury site. Each component is supplied as a freeze-dried powder that must be reconstituted with diluent prior to use. After reconstitution, each component is delivered by an application device to the wound site, at which time the components are mixed and clotting (glue formation) occurs. However, most freeze-dried preparations of fibrinogen require relatively long periods of time for rehydration, and may denature or aggregate to form insoluble particles. Thus a need exists for an improved method for stabilizing fibrinogen to minimize potential denaturation and aggregation of the protein and reduce the rehydration time.

### SUMMARY OF THE INVENTION

The present invention provides a process for stabilizing lyophilized blood proteins. In one embodiment, the invention is directed to a method for stabilizing lyophilized blood proteins, particularly lyophilized fibrinogen. The method comprises providing an aqueous solution of a blood protein. Hydroxypropyl-α-cyclodextrin is added to the solution in an amount sufficient to form a complex with at least a portion, and preferably all, of the blood protein. The solution is lyophilized to provide a dry blood protein/hydroxypropyl-α-cyclodextrin complex. The dry blood protein/hydroxypropyl-α-cyclodextrin complex may then be reconstituted to provide a solution of the blood protein, which can be administered to a patient.

It has been discovered that the stabilization of blood protein with hydroxypropyl-α-cyclodextrin prior to lyophilization can reduce denaturation of the protein during dry heat viral inactivation. Additionally, the reconstitution time for the lyophilized blood protein stabilized in accordance with practice of the present invention is substantially reduced.

### DETAILED DESCRIPTION

The present invention is directed to a method that incorporates the use of hydroxypropyl-α-cyclodextrin (HPαCD) to stabilize lyophilized proteins, particularly fibrinogen, and to enhance reconstitution of these proteins. The method comprises providing an aqueous solution of a blood protein. HPαCD is added to the solution in an amount sufficient to form a complex with at least part, and preferably all, of the blood protein. The complex is lyophilized to provide a dry blood protein/HPαCD complex. The dry blood protein/HPαCD complex may then be reconstituted to provide a solution of the blood protein, which can be administered to a patient.

Blood proteins with which the present process may be used include, but are not limited to, albumin, Factor II, Factor VII, Factor VIII, Factor IX, Factors X and Xₐ, fibrinogen, antithrombin III, transferrin, haptoglobin, gamma globulins, fibronectin, protein C, protein S and thrombin.

Cyclodextrins are homologous oligosaccharides that are obtained from starch by the action of enzymes from Bacillus macetans. α-Cyclodextrin is a cyclic molecule containing six α-D-glucopyranose units linked together at the 1,4 positions, as in amylose. This cyclic structure may also be referred to as a torus. HPαCD is commercially available from Cerestar USA, Inc. (Hammond, Ind.) or Pfanstiehl (Waukegan, Ill.).

The HPαCD may be added to an aqueous solution containing the blood protein before lyophilization at any suitable point in the purification process. Preferably, the HPαCD is added to an aqueous solution of the blood protein after all purification steps have been completed to prevent the HPαCD from forming a complex with impurities, which makes removal of the impurities more difficult.

If desired, the blood protein can be subjected to one or more viral inactivation steps prior to lyophilization, and preferably prior to complexing with the HPαCD. After lyophilization, preferably the blood protein is heated to a temperature and for a time sufficient to inactivate any viral contaminants. Preferably the complex is heated to a temperature of at least about 60° C., more preferably to at least about 80° C., still more preferably at least about 100° C., for a time of at least about 10 hours at 80° C. or at least about 1 hour at 100° C., and more preferably at least about 72 hours at 80° C. or at least about 3 hours at 100° C.

The blood protein can be subjected to a solvent detergent viral inactivation process instead of or in addition to viral inactivation by heat. Suitable solvent detergent viral inactivation processes are described in U.S. Pat. Nos. 4,540,573, and 4,764,369, the entire disclosures of which are incorporated herein by reference.

Preferably the HPαCD is added in an amount sufficient to assure the formation of a complex with all of the desired blood protein. More preferably the HPαCD is added in an amount such that the aqueous solution has a HPαCD concentration of at least about 0.5% weight per volume (wt/vol.), preferably from about 0.5% to about 15% wt/vol., and more preferably from about 1% to about 12% wt/vol. More particularly, when the blood protein is fibrinogen, preferably the HPαCD is added in an amount such that the aqueous solution has a HPαCD concentration of at least about 0.5% wt/vol., preferably from about 0.5% to about 4% wt/vol., and more preferably from about 1% to about 2.5% wt/vol.

It has been found that the presence of HPαCD substantially decreases the reconstitution time of the lyophilized blood protein. The time for reconstituting the lyophilized protein/hydroxypropyl-α-cyclodextrin complex, compared to the time for reconstituting a similar protein solution not containing hydroxypropyl-α-cyclodextrin, is preferably decreased by at least about 50%, more preferably by at least about 75%, still more preferably by at least about 90%, and even more preferably by at least about 95%.

If desired, an additional stabilizing agent can be included with the HPαCD to further reduce the reconstitution time. Examples of such agents include lysine and polysorbate 80 (Tween 80).

### EXAMPLE 1

Fibrinogen is manufactured from pooled cryo-poor and/or PTC-poor human plasma maintained at a temperature of 1.5±1.5° C. The pH is adjusted to 7.0±0.2 with either 1 M sodium bicarbonate or pH 4.0 acetate buffer. Sufficient cold SD3A ethanol is added to bring the plasma to a final alcohol concentration of 8%. During the alcohol addition, the temperature is gradually lowered to −2±4° C. The precipitate that forms (Fraction I precipitate) is removed by centrifugation at −2±1° C.

The Fraction I precipitate is extracted with about 9±2 kg of extraction buffer (0.40±0.15 M 6-amino-n-hexanoic acid; 0.05±0.01 M sodium citrate; 0.08±0.02 M sodium chloride; 7±4 units/mL heparin; pH 6.4±0.3) per kg of Fraction I preciptitate at pH 6.4±0.3. Reconstitution of the Fraction I precipitate is performed at 30±4° C. and yields Fraction I Solution The pH of Fraction I Solution is adjusted to 6.6+0.3 if necessary. The extracted Fraction I solution is clarified by centrifugation and/or filtration at 28±6° C. to produce Fraction I Filtrate.

Each kilogram of Fraction I Filtrate is mixed with 0.11±0.03 kg of Solvent Detergent Solution (3+0.5% tri-n-butyl phosphate; 10±1% polysorbate 80; water for injection) to a final concentration of0.30±0.1% tri-n-butyl phosphate and 1±0.3% polysorbate 80, and the pH of the mixture is adjusted to 6.6±0.3. The solution is mixed for 1 hour at 27±3° C. and transferred for further processing to a virally controlled area. Mixing is continued in the virally controlled area for an additional 6±1 hours at 27±3° C. The pH is adjusted as necessary to 6.6±0.3 during incubation.

The solution is cooled to 23±4° C., and the pH is adjusted to 6.8±0.3 with 1 N sodium hydroxide or 1 N acetic acid. The pH adjusted solution is cooled to 9±3° C. with constant mixing, and solid glycine is added (135±25 g per kg of pH adjusted solution). Mixing is continued for not less than 30 minutes at 3 to 11° C. to obtain a First Glycine Precipitate. The First Glycine Precipitate is removed from the suspension by centriguation or filtration and may be held frozen prior to further processing.

The First Glyine Precipitate is solubilized in approximately 9±2 kg of citrate saline buffer (0.02±0.005M sodium citrate; 0.12±0.03 M sodium chloride; pH 7.7±0.5) per kg of precipitate by mixing for at least 30 minutes at 30±4° C. The First Glycine Precipitate suspension is cooled to 23±4° C., and the pH is adjusted to 6.8±0.3 with 1 N acetic acid or 1 N sodium hydroxide. The adjusted solution is cooled to 9±3° C. with constant mixing.

Solid glycine is added to the pH adjusted solution (128±20 g per kg of adjusted solution) with vigorous mixing of the solution and care to prevent foaming. Mixing of the solution is continued for not less than 30 minutes at 3 to 11° C. to obtain a Second Glycine Precipitate. The Second Glycine Precipitate is removed from the suspension by centrifugation or filtration and may be held frozen prior to further processing.

The Second Glycine Precipitate is solubilized in about 9±2 kg of citrate saline buffer (0.02±0.005 M sodium citrate; 0.12±0.03M sodium chloride) per kg of precipitate by mixing continuously at 30±4° C. The Second Glycine Precipitate solution is kept at 30±4° C. The pH of the Second Glycine Precipitate solution is adjusted to 6.8±0.3 if necessary with 1 N acetic acid or 1 N sodium hydroxide.

The procedure for preparing the Second Glycine Precipitate is repeated to obtain a Third Glycine Precipitate. The Third Glycine Precipitate solution is clarified by centrifugation and/or filtration at a temperature of 27±7° C.

EXAMPLE 2

Fibrinogen preparations were prepared generally as set forth in Example 1. 10 kg of the Third Glycine Precipitate were mixed with a 4:1 ratio of buffer containing 0.02M sodium citrate, 0.12M sodium chloride, 3.2% sucrose, pH 6.7 at 30° C. until in solution. Insoluble material was removed by centrifugation. The solution was diafiltered against citrate-saline buffer and concentrated to about 3% fibrinogen. The solution was sterile filtered, filled into vials (50 mL in 100 mL vials), freeze-dried and stored in the lyophilized state. A number of vials of the lyophilized preparation were reconstituted with 50 mL of water and the contents pooled. The pooled material was diafiltered against a standard formulation buffer containing 8 mM sodium citrate, 50 mM Tris, 80 mM NaCl, 50 mM glycine, pH 6.7. The solution was concentrated to about 2.3% fibrinogen and aliquoted. Appropriate amounts of stock excipient solutions were added to each aliquot to obtain the final excipient concentrations shown in Table I. The solutions containing added excipients were filled into vials (8 mL in a 20 mL vial) and lyophilized. Following lyophilization, some of the vials were heated at 80° C. for 72 hours to inactivate viruses, and the vial contents were then reconstituted with 2 mL of water, where the reconstitution times and fibrinogen concentrations are set forth in Table I below.

TABLE I
RECONSTITUTION TIMES (MINUTES) OF FIBRINOGEN
PRODUCT FORMULATED WITH VARIOUS EXCIPIENTS
IN STANDARD BUFFER

| | | Ex. 2A | Ex. 2B |
|---|---|---|---|
| EXCIPIENT | Fill Conc (%) | 5% Protein | 6.5% Protein |
| No excipient | | >30, >30 | >30 |
| Tween 80 | 0.01 | | 9, 13, 5 |
| | 0.005 | | 43, 24 |
| Tween 80/Sucrose | 0.01/1 | | 9, 8 |
| | 0.005/1 | | 21, 22 |
| Tween 80/Lys | 0.01/1 | | 6, 3, 6, 5 |
| | 0.005/1 | | 12, 13, 14 |
| Tween 80/Lys/Sucrose | 0.01/1/1 | | 12, 11 |
| | 0.005/1/1 | | 27, 15 |
| HPβCD (Cerestar) | 1.0 | 4, 4 | 10, 9 |
| HPαCD | 1.0 | 2, 2 | 10, 12 |
| HPγCD | 1 | 1, 4 | 27, 16 |
| Hydroxyethyl β CD | 1 | 2, 4 | 14, 16 |
| Hydroxyethyl α CD | 1 | 10, 4 | 18, 20 |
| CarboxyMethyl β CD | 1 | 4, 6 | 15, 14 |
| Methyl β CD | 1 | 25, 4 | 17, 33 |
| Quaternary amine β CD | 1 | 24, 15 | 26, 28 |
| Quaternary amine γCD | 1 | 26, 15 | 27, 36 |
| Tertiary amine β CD | 1 | 6, 4 | >30, 18, 35 |
| Tertiary amine | 1 | 4, 7 | >30, 18, 19 |
| CarboxyMethyl β CD | | | |

EXAMPLE 3

A Third Glycine Precipitate fibrinogen preparation was prepared as described in Example 1. Portions of precipitates were resuspended with a 6:1 ratio of buffer containing 20 mM citrate and 124 mM sodium chloride, pH 6.7 at 30° C. until in solution. The solution was diafiltered against standard formulation buffer and concentrated to about 3% fibrinogen, and appropriate amounts of stock excipient solutions were added to obtain the final excipient concentrations shown in Tables IIA and IIB, below. The solutions containing added excipients were filled into vials (9.0 to 15.0 mL in a 20 mL vial) and lyophilized. Following lyophilization, some of the vials were heated at 80° C. for 72 hours to inactivate viruses, and the vial contents were then reconstituted with ⅓ to ½ the fill volume of water, where the reconstitution times and protein concentrations are set forth in Tables IIA and IIB. Other vials were also reconstituted with water at ⅓ to ½ the original fill volume without heating, where the reconstitution times and fibrinogen concentrations are set forth in Table III below.

TABLE IIA
RECONSTITUTION TIMES (MINUTES) OF FIBRINOGEN
PRODUCT FORMULATED WITH VARIOUS EXCIPIENTS
IN STANDARD BUFFER

| | | | Ex. 3B |
|---|---|---|---|
| | | Ex. 3A | 7.4% |
| EXCIPIENT | Fill Conc (%) | 6.5% Protein | Protein |
| No excipient | | >40 | Not Done |
| Tween 80 | 0.04 | | >30, >30, 29, 28 |
| | 0.02 | 9, 9, 15, 12, 8, 14, 25, 16 | |
| | 0.01 | 18, 20, 25, 19, >30, >30 | |
| Tween 80/Lysine | 0.04/1 | 7, 7, 4, 5 | 14, 18, 10 |
| | 0.02/1 | 15, 21, 9 | >30, >30 |
| Albumin | 1.5 | >35, 43, 48 | |
| | 1.0 | 33, 18, 27 | |
| HPβCD (Cerestar) | 1.5 | 10, 13, 4 | |
| HPβCD/Tween 80 | 1/.02 | | 24, 23 |
| HPαCD | 1.5 | 13, 5, 14, 13 | |
| HPαCD/Tween 80 | 1.5/2.0 | | 18, 18 |
| HPγCD | 1.5 | 26, 20 | |
| HPγCD/Tween 80 | 1.5/.02 | | 22, 25, 20 |
| Hydroxyethyl β CD | 1.5 | 28, >30 | |
| Hydroxyethyl α CD | 1.5 | >30, >30 | |
| CarboxyMethyl β CD | 1.5 | 12, 12, 29, 18, 19 | |
| CarboxyMethyl βCD/Tween 80 | 1.5/.02 | | 21, 15, 17 |

TABLE IIB
RECONSTITUTION TIMES (MINUTES) OF FIBRJNOGEN PRODUCT FORMULATED
WITH VARIOUS EXCIPIENTS IN STANDARD BUFFER

| | Fill conc | Ex. 3C | Ex. 3D | Ex. 3E | Ex. 3F |
|---|---|---|---|---|---|
| EXCIPIENT | (%) | 7% Protein | 6.5% Protein | 7% Protein | 7.5% Protein |
| No excipient | | | >100, >100 | | >18 hours |
| Tween | 0.16/1 | | | 42, 18, 31, 40 | 24, 26, 24, 21 |
| 80/Lys | 0.08/1 | | 4, 6, 14, 7 | | |
| | 0.04/1 | 40, 36, 39, 15, | 4, 10, 23, 12 | | |
| | | 17, 22, 42, 20 | | | |
| Albumin/ | 1.0/.16 | | | >24 hours | |
| Tween 80 | | | | | |
| HPβCD | 3.0 (Janssen) | | 21, 32, 35 | | |
| | 2.5 (Cerestar) | | | 38, 20, 24, 37, 40 | 18, 21, 9, 16, 25 |
| | 2.0 (Janssen) | | 24, 43, 32 | | |
| HPβCD | 1.5 (Janssen) | | 20, 20, 31, 19 | | |
| | 1.5 (Cerestar) | 13, 11, 12, 13, | 12, 14, 11 | | |
| | | 13, 12, 7, 8 | | | |
| HPαCD | 4 | | | 11, 9, 10, 14, 15 | 14, 17, 13, 17, 11 |
| | 2.5 | | | 44, 28, 38, 30 | 12, 18, 10, 16, 17 |
| | 1.5 | 35, 29, 34, 27, | 26, 37, 29, 32 | 45, 50 | |
| | | 31, 24, 16, 30, 21 | | | |
| HPαCD/ | 2.5/0.16 | | | 22, 35, 39, 23 | |
| Tween 80 | | | | | |
| HPαCD/Lys | 4.0/1 | | | | 7, 4, 4, 10, 10, |
| | 2.5/1 | | | 8, 9, 10, 7, 8 | 16, 13, 10, 14, |
| | | | | | 10, 10 |
| HPαCD/Lys/ | 4.0/1/0.16 | | | | 7, 8, 10, 12 |
| Tween 80 | | | | | |
| HPγCD | 4 | | | 87, 110 | |
| Hydroxeythyl α | 4 | | | 41, 68 | |
| CD | | | | | |
| CarboxyMethyl | 4 | | | 97, 46, 3 5 | |
| β CD | | | | | |
| Tertiary amine | 4 | | | 67 | |
| β CD | | | | | |
| Tertiary amine | 4 | | | 99 | |
| CarboxyMethyl | | | | | |
| β CD | | | | | |

TABLE III
Fibrinogen Formulations and Reconstitution Times
for Non Heat-Treated Fibrinogen Vials

| | | Excipient | Fill Vol. | Reconst. | Fibrin. | Reconst. |
|---|---|---|---|---|---|---|
| | Excipient | Conc. | (mL) | Vol. (mL) | (mg/mL) | Time |
| Ex. 3G | HPαCD | 1.5% | 12.2 | 5 | ND | 16 min. |
| | HPαCD | 1.5% | 10.6 | 5 | ND | 17 min. |
| | HPβCD | 1.5% | 12.2 | 5 | ND | 10 min. |
| | HPβCD | 1.5% | 10.6 | 5 | ND | 3 min. |
| Ex. 3H | HPαCD | 1.5% | 13 | 5 | 67 | 17, 19 min. |
| | HPβCD | 1.5% | 13 | 5 | 64 | 11, 12 min. |
| | No Excipient | N/A | 13 | 5 | 66 | >24 h, >24 h |
| Ex. 3I | HPαCD | 1.5% | 12.5 | 5 | 64 | 39 min. |
| | HPαCD | 2.5% | 12.5 | 5 | 69 | 23, 33 min. |
| | HPαCD | 4% | 12.5 | 5 | 73 | 11.5, 13 min. |
| | HPβCD | 2.5% | 12.5 | 5 | 69 | 21, 7 min. |
| | No Excipient | N/A | 12.5 | 5 | ND | >7.5 h |
| Ex.3J | HPαCD | 2.5% | 15 | 5 | 76 | 11, 15 min. |
| | HPαCD | 4% | 15 | 5 | 77 | 11.5, 15 min. |
| | HPβCD | 2.5% | 15 | 5 | 79 | 11, 11 min. |
| | No Excipient | N/A | 15 | 5 | 81 | >24 h, >24 h |

N/A = Not Applicable; ND = Not Determined

### EXAMPLE 4

A Third Glycine Precipitate fibrinogen preparation was prepared as described in Example 1. Portions of precipitates were resuspended with a 6:1 ratio of buffer containing 20 mM citrate and 124 mM sodium chloride, pH 6.7 at 30° C. until in solution. The solution was diafiltered against standard formulation buffer and concentrated to about 3% fibrinogen, and appropriate amounts of stock excipient solutions were added to obtain the final excipient concentrations shown in Table IV, below. The solutions containing added excipients were filled into vials (15.0 mL in a 20 mL vial) and lyophilized. Following lyophilization, some of the vials were heated at 80° C. for 72 hours to inactivate viruses, and the vial contents were then reconstituted with water at ⅓ to ½ the original fill volume, where the reconstitution times and protein concentrations are set forth in Table IV. Other vials were not heat treated and vial contents were reconstituted with 5 mL of water, where the reconstitution times and fibrinogen concentrations are set forth in Table V.

TABLE IV
RECONSTITUTION TIMES (MINUTES) AFTER HEAT TREATMENT OF
FIBRINOGEN PRODUCT FORMULATED WITH VARIOUS EXCIPIENTS
IN STANDARD BUFFER*

| | | Ex. 4A | Ex. 4B | Ex. 4C | | |
|---|---|---|---|---|---|---|
| | Fill | 7.4% | 6.4% | 8.6% | | |
| EXCIPIENT | Conc (%) | Protein | Protein | Protein | Ex. 4D | Ex. 4E |
| No excipient | | >30, >30 | | | | |
| HPαCD | 4.0 | 10, 6, 6 | 5, 5, 6, 6, 2 | 7, 6, 6 | 8, 8, 7 | 10, 10 |
| | 2.5 | 7, 5, 9 | 5, 6, 7, 5, 5 | | | |
| HPαCD/Lys | 4.0/1 | 3, 4, 4, 2, 2, 5 | 4, 4, 6, 6 | 4, 4, 5 | 5, 2 | |
| | 2.5/1 | 5, 5, 7, 3, 4, 3 | 5, 5, 2, 8, 6 | | | |
| Lys | 1.0 | >30, >30, >30 | | | | |
| HPβCD | 2.5 | 7, 6, 8 | 14, 12 | | | |
| | 1.5 | 5, 9, 17 | 11, 10, 11 | | | |

*All vials were filled with 13.6 to 15 mL of product and reconstituted with 5 mL of sterile water.

TABLE V
Fibrinogen Formulations and Reconstitution Times
for Non Heat-Treated Fibrinogen Vials

| | | Excipient | Fill Vol. | Recoust. | Fibrin. | Reconst. |
|---|---|---|---|---|---|---|
| | Excipient | Concen. | (mL) | Vol. (mL) | (mg/mL) | Time |
| Ex.4F | HPαCD | 2.5% | 15 | 5 | 84 | 10, 6 min. |
| | HPαCD | 4% | 15 | 5 | 82 | 3, 4 min. |
| | HPβCD | 2.5% | 15 | 5 | 82 | 4.5, 7 min. |
| | HPβCD | 1.5% | 15 | 5 | 84 | 8.5, 12 min. |
| | No Excipient | N/A | 15 | 5 | 83 | >60, >80 min. |
| Ex. 4G | HPαCD | 2.5% | 15 | 5 | 69 | 5.5, 7, 6, 9 min. |
| | HPαCD | 4% | 15 | 5 | 63 | 4, 6, 4, 5, 5 min. |
| | HPβCD | 2.5% | 15 | 5 | 66 | 10, 7 min. |
| | HPβCD | 1.5% | 15 | 5 | 66 | 7.5, 9 min. |
| | No Excipient | N/A | 15 | 5 | ND | >2 h, >4 h |
| Ex. 4H | HPαCD | 4% | 15 | 5 | ND | 4, 7 min. |
| Ex. 4I | HPαCD | 4% | 15 | 5 | ND | 9, 6 min. |
| Ex. 4J | HPαCD | 4% | 15 | 5 | ND | 15 min. |

N/A = Not Applicable
ND = Not Determined

The above descriptions of exemplary embodiments of processes for preparing stabilized fibrinogen products are for illustrative purposes. Because of variations that will be apparent to those skilled in the art, the present invention is not intended to be limited to the particular embodiments described above. This invention can also be practiced in the absence of any element not specifically disclosed. The scope of the invention is described in the following claims.

What is claimed is:

1. A process for stabilizing a blood protein solution comprising:
(a) providing a blood protein solution;
(b) adding to the solution hydroxypropyl-α-cyclodextrin in an amount sufficient to form a stable complex with the protein; and
(c) lyophilizing the solution of step (b) to form a lyophilized protein/hydroxypropyl-α-cyclodextrin complex.

2. The process according to claim 1, further comprising reconstituting the lyophilized protein/hydroxypropyl-α-cyclodextrin complex.

3. The process according to claim 1, further comprising heating the blood protein solution, before or after adding hydroxypropylα-cyclodextrin, at least about 60° C. for a time sufficient to inactivate any viruses present in the protein/hydroxypropylα-cyclodextrin complex.

4. The process according to claim 3 wherein the blood protein solution is heated for at least about 10 hours.

5. The process according to claim 3 wherein the blood protein solution is heated to a temperature of at least about 80° C. for at least about 72 hours.

6. The process according to claim 3 wherein the blood protein solution is heated to about 100° C. for at least about 1 hour.

7. The process according to claim 1, further comprising subjecting the blood protein solution, before or after adding the hydroxypropyl-α-cyclodextrin, to a solvent detergent viral inactivation step.

8. The process according to claim 1, wherein the hydroxypropyl-α-cyclodextrin is present in the protein solution in an amount ranging from about 0.5% wt/vol. to about 15% wt/vol.

9. The process according to claim 1, wherein the hydroxypropyl-α-cyclodextrin is present in the protein solution in an amount ranging from about 1% wt/vol. to about 12% wt/vol.

10. The process according to claim 2, wherein the protein is present in the reconstituted protein/hydroxypropyl-α-cyclodextrin complex in an amount greater than about 0.1% wt/vol.

11. The process according to claim 2 wherein the protein is present in the reconstituted protein /hydroxypropyl-α-cyclodextrin complex in an amount from about 1% to about 8%.

12. The process according to claim 1 wherein the protein is selected from the group consisting of albumin, Factor II, Factor VII, Factor VIII, Factor IX, Factors X and Xₐ, fibrinogen, antithrombin III, transferrin, haptoglobin, gamma globulins, fibronectin, protein C, protein S, thrombin and C1-inhibitor.

13. The process according to claim 1, wherein the protein is fibrinogen.

14. The process according to claim 12, wherein the hydroxypropyl α-cyclodextrin is present in the protein solution in an amount ranging from about 0.5% wt/vol. to about 15% wt/vol.

15. The process according to claim 12, wherein the hydroxypropyl-α-cyclodextrin is present in the protein solution in an amount ranging from about 2% wt/vol. to about 12% wt/vol.

16. The process according to claim 12, wherein the fibrinogen is present in the reconstituted protein/hydroxypropyl-α-cyclodextrin complex in an amount greater than about 1% wt/vol.

17. The process according to claim 12, wherein the protein is fibrinogen, and the fibrinogen is present in the reconstituted protein /hydroxypropyl-α-cyclodextrin complex in an amount from about 3% wt/vol. to about 10% wt/vol.

18. A process for stabilizing a fibrinogen solution comprising:
(a) providing a fibrinogen solution;
(b) adding to the solution hydroxypropyl-α-cyclodextrin in an amount sufficient to form a stable complex with the protein;
(c) lyophilizing the solution of step (b) to form a lyophilized fibrinogen/hydroxypropyl-α-cyclodextrin complex; and
(d) reconstituting the lyophilized fibrinogen/hydroxypropyl-α-cyclodextrin complex.

19. A lyophilized blood protein/hydroxypropyl-α-cyclodextrin complex prepared by:
(a) providing a blood protein solution;
(b) adding to the solution hydroxypropyl-α-cyclodextrin in an amount sufficient to form a stable complex with the protein; and
(c) lyophilizing the solution of step (b) to form the lyophilized blood protein/hydroxypropyl-α-cyclodextrin complex.

20. A blood protein product prepared by:
(a) providing a blood protein solution;
(b) adding to the solution hydroxypropyl-α-cyclodextrin in an amount sufficient to form a stable complex with the protein;
(c) lyophilizing the solution of step (b) to form a lyophilized protein/hydroxypropyl-α-cyclodextrin complex; and
(d) reconstituting the lyophilized protein/hydroxypropyl-α-cyclodextrin complex.

21. A fibrinogen product prepared by:
(a) providing a fibrinogen solution;
(b) adding to the solution hydroxypropyl-(α-cyclodextrin in an amount sufficient to form a stable complex with the protein;
(c) lyophilizing the solution of step (b) to form a lyophilized fibrinogen/hydroxypropyl-α-cyclodextrin complex; and
(d) reconstituting the lyophilized fibrinogen/hydroxypropyl-α-cyclodextrin complex.

22. A blood protein product comprising a lyophilized solution of a stable complex of protein and hydroxypropyl-α-cyclodextrin.

23. The product according to claim 22, wherein the hydroxypropyl-α-cyclodextrin is present in the solution in an amount ranging from about 0.5% wt/vol. to about 15% wt/vol.

24. The product according to claim 22, wherein the hydroxypropyl-α-cyclodextrin is present in the solution in an amount ranging from about 1% wt/vol. to about 12% wt/vol.

25. The product according to claim 22, wherein the blood protein is fibrinogen.

26. A stabilized blood protein solution comprising a complex of the blood protein and hydroxypropyl-α-cyclodextrin.

27. The solution according to claim 26, wherein the protein is present in the complex in an amount greater than about 3% wt/vol.

28. The product according to claim 26, wherein the hydroxypropyl-α-cyclodextrin is present in the solution in an amount ranging from about 0.5% wt/vol. to about 15% wt/vol.

29. The process according to claim 26, wherein the hydroxypropyl-α-cyclodextrin is present in the solution in an amount ranging from about 1% wt/vol. to about 12% wt/vol.

30. The product according to claim 26, wherein the blood protein is fibrinogen.
